(19) United States
(12) Patent Application Publication
(10) Pub. No.: US 2008/0082172 A1
(43) Pub. Date: Apr. 3, 2008

(54) Interspinous process spacer

(76) Inventor: Roger P. Jackson, Prairie Village, KS (US)

Correspondence Address: John C. McMahon, PO Box 30069, Kansas City, MO, 64112, US

(21) Appl. No.: 11/801,191

(22) Filed: May 9, 2007

Related U.S. Application Data
(60) Provisional application No. 60/848,278, filed on Sep. 29, 2006.

(51) Int. Cl. A61F 2/44; A61B 17/10; A61B 17/58

(52) U.S. Cl. 623/17.16; 606/139; 606/72

(57) ABSTRACT

An interbody or intervertebral spacer device for placement in an interspinous space between a pair of adjacent interspinous processes includes arcuate opposed abutment surfaces sized and shaped for engaging adjacent spinal processes. A curved through channel is disposed between the opposed abutment surfaces. A pair of bands is receivable in the curved through channel. A method of implanting such an interspinous spacer includes the steps of making an incision lateral to the spine; laterally inserting the spacer between a pair of interspinous processes without detachment of the supraspinous ligament; and laterally securing the spacer to the pair of interspinous processes with a pair of bands.

### CROSS REFERENCE TO RELATED APPLICATIONS

This application claims the benefit of U.S. Provisional Application No. 60/848,278 filed Sep. 29, 2006.

### BACKGROUND OF THE INVENTION

The present application is directed to an interbody device for implantation between a pair of adjacent interspinous processes for the purpose of providing dynamic support between the vertebrae, and more particularly, to such an intervertebral implant device that is implanted by a lateral minimally invasive method.

In the human spine, the pad or disc between vertebrae can become damaged and deteriorate due to injury, disease or other disorders. Upon such an occurrence, the discs may narrow or flatten, resulting in painful mechanical instability. With particular reference to the lower back, when the disc deteriorates, narrowing and bulging of the disc occurs, causing the two vertebrae that are separated by the disc to move toward one another. This may cause entrapment of nerve roots and resulting pain to the patient.

In an attempt to relieve such lower back pain, intervertebral implants have been designed that include a spacer inserted between the spinous processes. It is known to hold such spacers by ties or bands wrapped around adjacent spinous processes. Such implants advantageously support and limit the movement of the vertebrae treated and yet are not permanently fixed to the vertebral bone, thus avoiding loosening and rigidity issues prevalent in the more permanent bone fixing systems. The non-rigid, removable fixation provided by interspinous spacers is particularly advantageous, for example, for younger patients needing to manage pain during initial forms of degenerative intervertebral lumbar disc disease, and in some older patients with spinal stenosis and/or degenerative spondylolisthesis.

However, one of the drawbacks to such interspinous spacers is that implantation of the spacer between a pair of adjacent spinous processes requires making an incision centrally along the spine followed by detachment or resection of the supraspinous ligament from at least the two adjacent spinous processes and also resection of the interspinous ligament between the two spinous processes. Detachment and resection of ligaments is not desirable as it is invasive to the patient and extends healing time. In particular, the supraspinous ligament is thicker and broader in the lumbar than in the thoracic region, making such a procedure even more undesirable in the lower back region. It is thus desirable to develop interspinous process spacers and methods of implantation that are less invasive to the patient, preferably such spacers and methods of use that do not require detachment or removal of the supraspinous and interspinous ligaments and that can be inserted laterally from only one side.

### SUMMARY OF THE INVENTION

An interbody or intervertebral spacer device for placement in an interspinous space between a pair of adjacent interspinous processes includes arcuate opposed abutment surfaces sized and shaped for engaging adjacent spinal processes. A curved through channel is disposed between the opposed abutment surfaces. A pair of bands is receivable in the curved through channel.

The illustrated device further includes at least one arcuate furrow disposed in one of the abutment surfaces. In a particular embodiment, one of the abutment surfaces has a centrally located arcuate furrow running substantially perpendicular to the channel and the other surface includes a notch.

A method of implanting an interspinous spacer according to the invention includes the steps of making an incision lateral to the spine; laterally inserting the spacer between a pair of interspinous processes without detachment of the supraspinous ligament; and laterally securing the spacer to the pair of interspinous processes with a pair of bands.

### OBJECTS AND ADVANTAGES OF THE INVENTION

Therefore, it is an object of the present invention to overcome one or more of the problems with intervertebral spacers described above. Further objects of the present invention are: to provide an interspinous spacer device that may be implanted by a minimally invasive method; to provide such a device having a shape that is easily received in an interspinous space; to provide such a device including bands, tapes or ties for attachment to the interspinous process; to provide such a device having apertures and/or contours for supporting such bands, tapes or ties; to provide such a device that may be firmly secured between two spinous processes; to provide such a device that may cooperate with at least one other such device for use on either side of a central spinous process; to provide such a device that exhibits strong structural integrity; to provide such a device with sufficient compression strength to ensure a long life span; to provide such a device having a compact structure with a reduced volume and weight; to provide such a device designed to promote ease of installation; to provide such a device that may be implanted without sectioning or detaching the supraspinous ligament; to provide such a device that may be implanted without resecting the interspinous ligament; and to provide such a device that is relatively easy to construct, inexpensive to produce and especially well-suited for the intended usage thereof.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is an enlarged perspective view of an interspinous spacer of the invention.

FIG. 2 is a rear elevational view of the spacer of FIG. 1.

FIG. 3 is a top plan view of the spacer of FIG. 1.

FIG. 4 is a cross-sectional view, taken along the line 4-4 of FIG. 3.

FIG. 5 is a cross-sectional view taken along the line 5-5 of FIG. 2.

FIG. 6 is an enlarged partial perspective and generally schematic view of a patient's spine showing an early stage of performing an incision in a process according to the invention.

FIG. 7 is an enlarged partial perspective and generally schematic view similar to FIG. 6, showing a preparation stage of a process according to the invention subsequent to the stage shown in FIG. 6.

FIG. 8 is an enlarged perspective view of an interspinous trial spacer.

FIG. 9 is a front elevational view of the trial spacer of FIG. 8.

FIG. 10 is a cross-sectional view taken along the line 10-10 of FIG. 9.

FIG. 11 is a top plan view of the trial spacer of FIG. 8.

FIG. 12 is an enlarged exploded partial perspective view of the trial spacer of FIG. 8 shown with a snap-on tool.

FIG. 13 is an enlarged partial side elevational view of the spacer and tool of FIG. 12 with portions broken away to show detail thereof.

FIG. 14 is an enlarged and partial view, similar to FIG. 7, showing insertion of the trial spacer and tool of FIGS. 12 and 13 according to a method of the invention.

FIG. 15 is an enlarged and partial view, similar to FIG. 14, showing a subsequent step of a method according to the invention wherein the trial spacer is inserted into an interspinous space as a test to determine an appropriately sized spacer of FIG. 1.

FIG. 16 is a perspective view showing a pair of tools for use in processes of the invention.

FIG. 17 is a an enlarged and partial view, similar to FIG. 15, showing the incision and a portion of the patient's spine, and further showing an early stage of implantation of a band according to a method of the invention with one of the tools shown in FIG. 16.

FIG. 18 is an enlarged and partial top plan view of the patient's spine, band and tool shown in FIG. 17, further illustrating the lateral nature of the process step of FIG. 17, with portions of the spine that are not exposed by the incision being shown in phantom.

FIG. 19 is an enlarged and partial top plan view, similar to FIG. 18, showing a later stage in the band implantation process.

FIG. 20 is an enlarged and partial top plan view, similar to FIGS. 18 and 19, showing implantation of a second band according to a method of the invention.

FIG. 21 is an enlarged and partial top plan view, similar to FIG. 20, showing a later stage in the implantation of the second band.

FIG. 22 is an enlarged and partial perspective view, similar to FIG. 14, 15 and 17, showing both of the bands being implanted.

FIG. 23 is an enlarged and partial perspective view, similar to FIG. 22, showing a subsequent method step of threading of the spacer of FIG. 1 onto the bands.

FIG. 24 is an enlarged and partial perspective view, similar to FIG. 23, showing a subsequent method step of positioning the bands.

FIG. 25 is an enlarged and partial perspective view, similar to FIG. 24, showing a subsequent method step of inserting the spacer of FIG. 1 into an interspinous space.

FIG. 26 is an enlarged and partial top plan view, similar to FIGS. 18-21, showing a method step subsequent to the step shown in FIG. 25 wherein the bands are being secured about the spinous process by a pair of band holding and tensioning tools.

FIG. 27 is an enlarged and partial top plan view, similar to FIG. 26 showing the bands finally secured about the spinous process.

FIG. 28 is an enlarged and partial perspective view showing the band tightening process shown in FIGS. 26 and 27.

FIG. 29 is an enlarged and partial top plan view, similar to FIG. 18, showing an early stage in a process according to the invention of implanting at least two spacers of FIG. 1 wherein a first spacer is being threaded.

FIG. 30 is an enlarged and partial top plan view, similar to FIG. 29 showing a subsequent step of implantation of a band for the second spacer.

FIG. 31 is an enlarged and partial top plan view, similar to FIG. 30, showing a subsequent step of threading the second spacer.

FIG. 32 is an enlarged and partial top plan view, similar to FIG. 31, showing a subsequent step of band attachment and tightening.

FIG. 33 is an enlarged and partial perspective view showing the two spacers of FIG. 32 fully implanted with the bands tightened.

FIG. 34 is an enlarged perspective view of a second embodiment of an interspinous spacer of the invention.

FIG. 35 is a front elevational view of the spacer of FIG. 34.

FIG. 36 is a top plan view of the spacer of FIG. 34.

FIG. 37 is a cross-sectional view, taken along the line 37-37 of FIG. 36.

FIG. 38 is a cross-sectional view taken along the line 38-38 of FIG. 35, and also showing the spacer with portions of the spinous process and a first band attaching the spacer to the interspinous process.

FIG. 39 is an enlarged partial perspective and generally schematic view of a patient's spine showing an early stage of performing an incision in a process according to the invention.

FIG. 40 is an enlarged partial perspective and generally schematic view similar to FIG. 39, showing a preparation stage of a process according to the invention subsequent to the stage shown in FIG. 39.

FIG. 41 is an enlarged and partial view, similar to FIG. 40, showing insertion of a trial spacer and the tool of FIGS. 12 and 13 according to a method of the invention wherein the trial spacer is inserted in an interspinous space as a test to determine an appropriately sized spacer of FIG. 34.

FIG. 42 is a perspective view showing a pair of tools for use in processes of the invention.

FIG. 43 is a an enlarged and partial view, similar to FIG. 41, showing the incision and a portion of the patient's spine, and further showing an early stage of implantation of a band according to a method of the invention with one of the tools shown in FIG. 42.

FIG. 44 is an enlarged and partial top plan view of the patient's spine, band and tool shown in FIG. 43, further illustrating the lateral nature of the process step of FIG. 43, with portions of the spine that are not exposed by the incision being shown in phantom.

FIG. 45 is an enlarged and partial top plan view, similar to FIG. 44, showing a later stage in the band implantation process.

FIG. 46 is an enlarged and partial top plan view, similar to FIGS. 43 and 44, showing subsequent implantation of a second band according to a method of the invention.

FIG. 47 is an enlarged and partial top plan view, similar to FIG. 46, showing a later stage in the implantation of the second band.

FIG. 48 is an enlarged and partial perspective view showing both of the bands being implanted prior to threading of the spacer of FIG. 34.

FIG. 49 is an enlarged and partial perspective view, similar to FIG. 48, showing a subsequent method step of threading of the spacer of FIG. 34 onto the bands.

FIG. 50 is an enlarged and partial perspective view, similar to FIG. 49, showing a subsequent method step of inserting the spacer of FIG. 34 into an interspinous space.

FIG. 51 is an enlarged and partial top plan view, showing a method step subsequent to the step shown in FIG. 50 wherein the bands are being secured about the spinous process using a pair of band holding tools.

FIG. 52 is an enlarged and partial top plan view, similar to FIG. 51 showing the bands finally secured about the spinous process.

FIG. 53 is an enlarged and partial perspective view showing two spacers of FIG. 34 fully implanted with the bands tightened according to a process of the invention.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

It is also noted that any reference to the words top, bottom, up and down, and the like, in this application refers to the alignment shown in the various drawings, as well as the normal connotations applied to such devices, and is not intended to restrict positioning of the spacers in actual use. It is also noted that reference to words such as front, back, anterior and posterior used in this application also refer to the alignment shown in the various drawings, and in particular, when possible, with reference to the human spine and human body, but also is not intended to restrict positioning of the spacers in actual use.

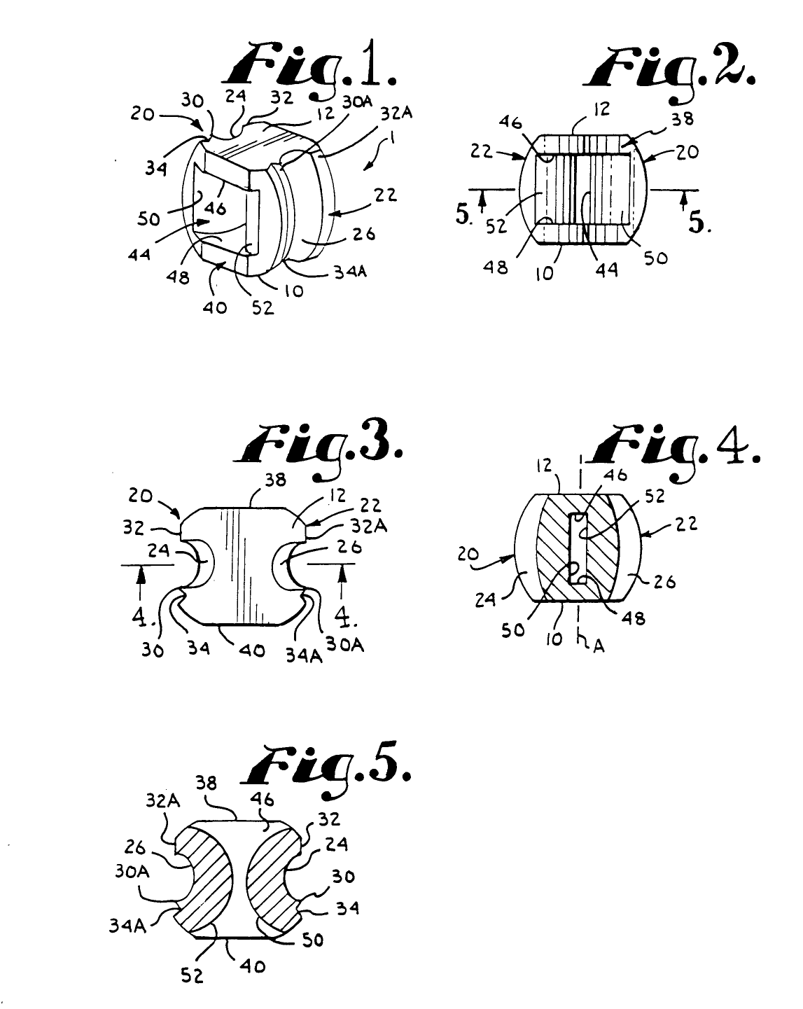

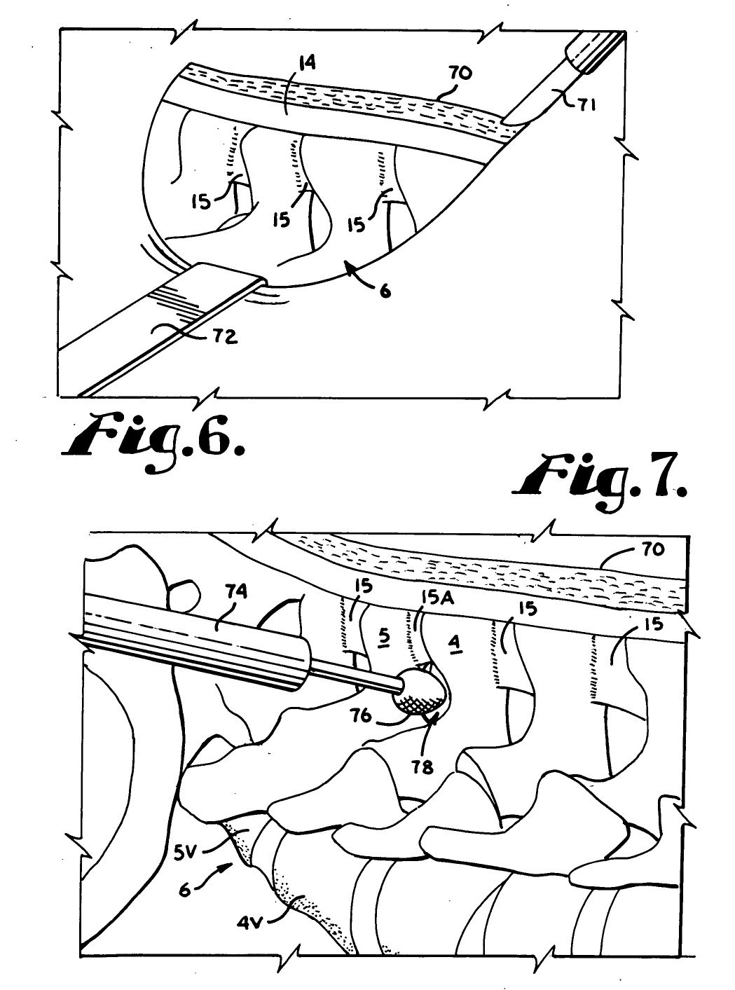

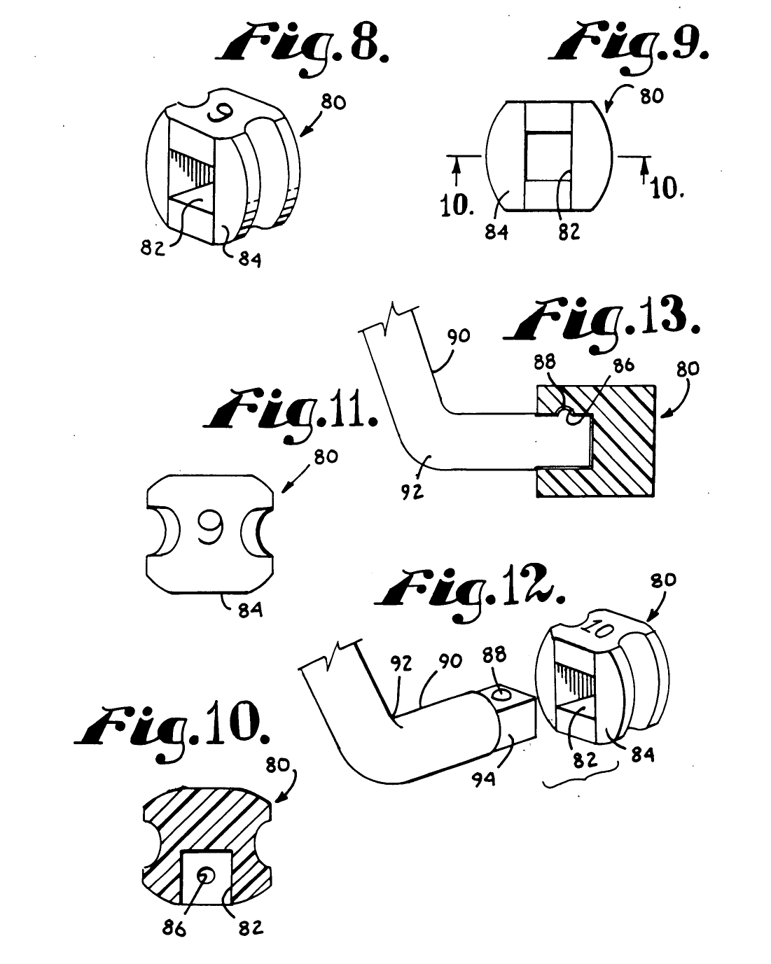

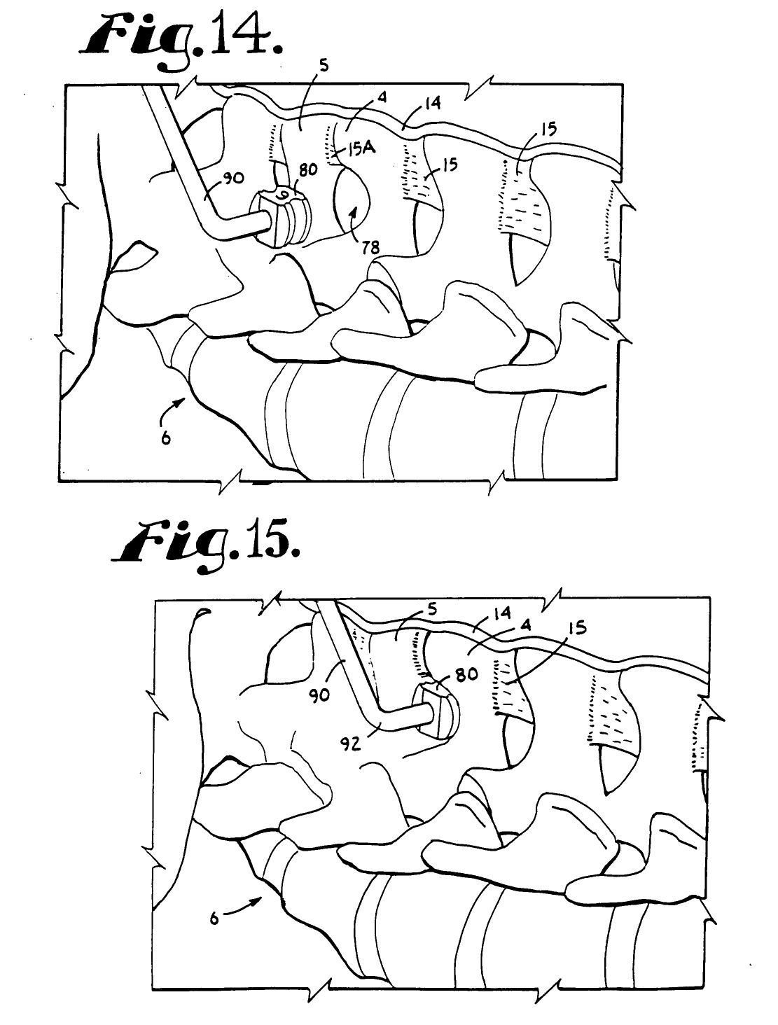

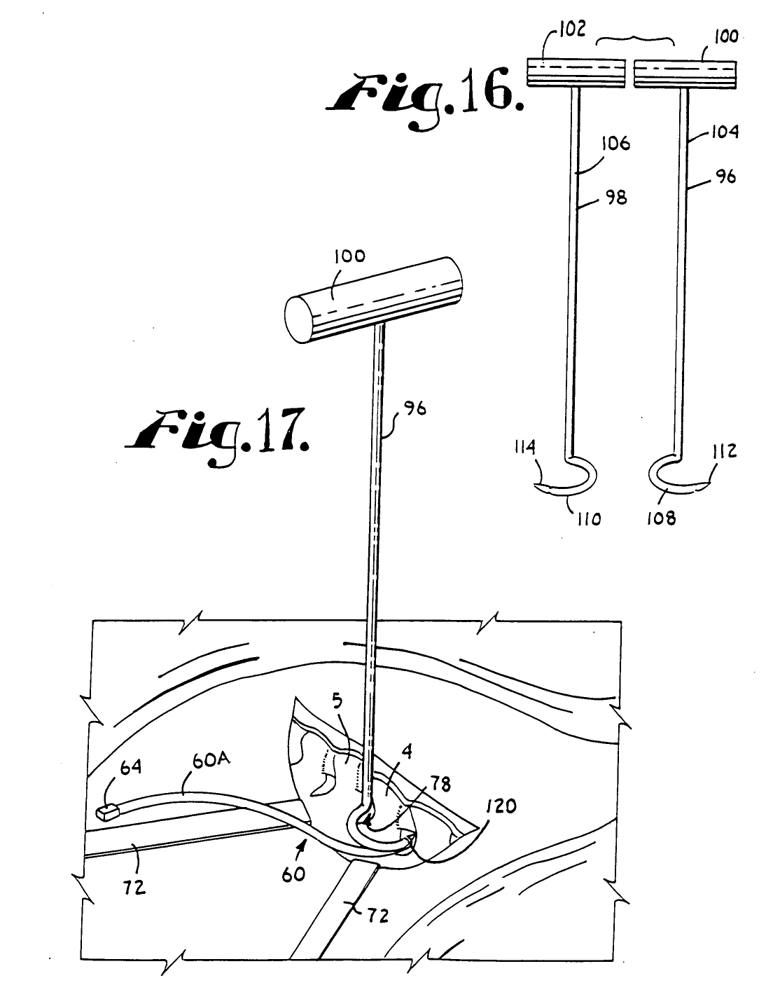

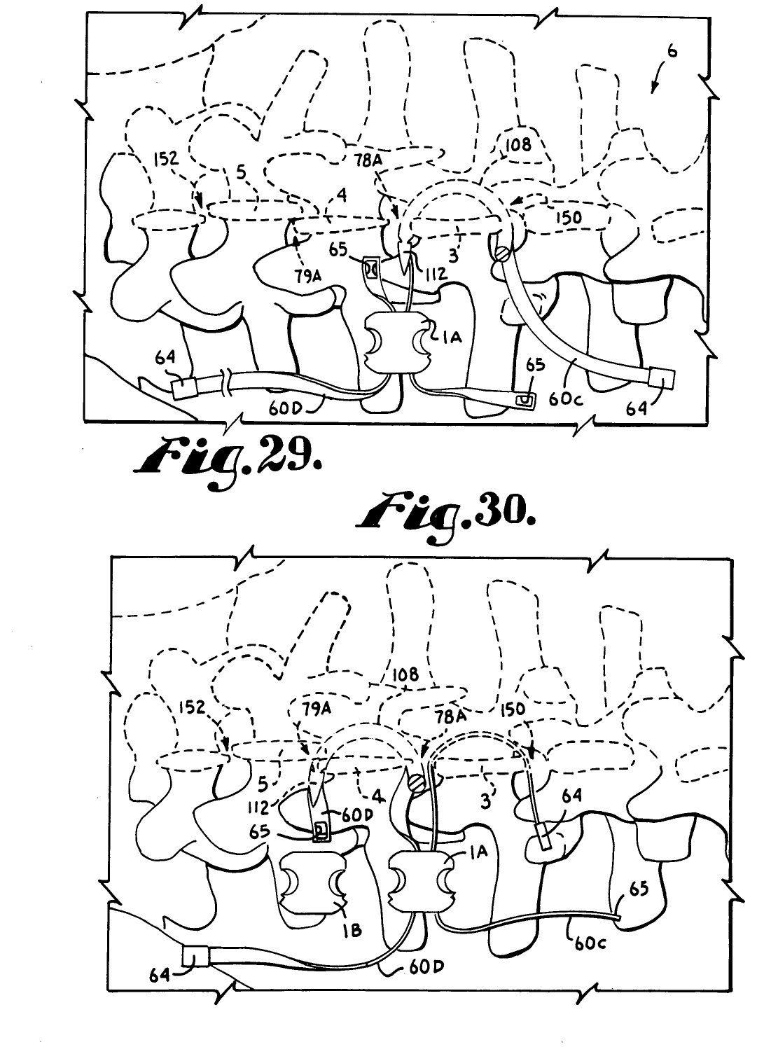

With reference to FIGS. 1-28, the reference numeral 1 generally designates an interspinous process spacer device of the present invention. The device 1 is used to maintain proper spacing between a pair of spinous processes 4 and 5 of a human spine, preferably a lower portion thereof, generally 6. The device 1 is preferably constructed from a single, unitary blank or molded, strong structure. The device 1 may be rigid or somewhat elastic and may be made from metals, metal alloys, plastics and/or composites. For example, the device 1 may be molded or machined from polymer or plastic materials such as polyetheretherketone (PEEK), silastics and polyurethanes. Devices 1 according to the invention are made in incremental sizes so that a desired size of device 1 is implanted for cooperating with a particular patient's spine 6.

In particular, the illustrated device 1 has a substantially flat anterior surface 10 for facing toward vertebrae 4V and 5V of the lower spinal portion 6 and a substantially flat posterior surface 12 opposite the surface 10 for facing away from the vertebrae and toward the supraspinous ligament 14 and a portion of interspinous ligament 15 that is disposed between the pair of spinous processes 4 and 5, the surface 12 being identical or substantially similar to the surface 10. The surfaces 10 and 12 are disposed in substantially parallel planes; however, such surfaces could be non-parallel.

The device 1 further includes an inferior or lower saddle-like abutment face or portion, generally 20, and an opposite substantially identical or similar superior or upper abutment face or portion, generally 22. The portions or faces 20 and 22 are arcuate having a substantially convex outer profile, curving substantially uniformly outwardly from the opposed surfaces 10 and 12. Formed in faces 20 and 22 are respective substantially uniform and centrally located U-shaped and arcuate furrows or channels 24 and 26 sized and shaped for engagement with the respective spinous processes 5 and 4 prepared for receiving the device 1 according to a method of the invention as will be described in greater detail below. The channels 24 and 26 are non-linear, having a substantially convex outer profile running from the surface 10 to the surface 12. The channels 24 and 26 are each defined in part by a pair of rims or ridges; the channel 24 being bounded by the rims or ridges 30 and 32 and the channel 26 being bounded by the rims or ridges 30A and 32A. Formed in the rims 30 and 30A are tool receiving grooves 34 and 34A, respectively. Although grooves 34 and 34A are shown that run from the surface 10 to the surface 12, it is foreseen that other types of tool receiving formations may be used including but not limited to notches, apertures and the like.

The rims 32 and 32A are adjacent to a lateral insertion or leading face or portion, generally 38 that is disposed opposite a lateral trailing face or portion, generally 40. The grooved rims 30 and 30A are disposed near the face 40. Both faces 38 and 40 are preferably beveled or rounded near the respective rims 30, 30A and 32 and 32A to aid in inserting the device 1 between the spinous processes 4 and 5. A band receiving channel 44 extends through the device 1 between the lateral faces 38 and 40. The channel is further defined by a pair of opposed substantially flat walls 46 and 48 and a pair of arcuate walls 50 and 52. The walls 46 and 48 are substantially parallel to one another and to the anterior and posterior surfaces 10 and 12. The walls 50 and 52 curve towards one another. As illustrated in FIG. 4, at a smallest opening in an interior of the device 1, the walls defining the channel 44 form an opening having a substantially rectangular cross-section, a length of the rectangular cross-section extending along a substantially central axis A. The channel 44 then widens in a direction toward the face 38 and also substantially the same in an opposite direction toward the face 44 as best illustrated in FIGS. 1, 2 and 5.

One or more devices 1 according to the invention are implantable between pairs of spinous processes attaching to adjacent spinous processes utilizing bands, ties or tapes, generally 60. The device 1 and attached bands provide for non-rigid stabilization without permanent fixation in the vertebral bone. The device 1 also relieves low-back pain due to disc pathology and symptoms from spinal stenosis caused by degenerative disc disease, spinal arthritis and instability, being useful as an initial substitute to pedicle screw fixation for dynamic spinal stabilization, total disc prostheses and spinal fusion methods.

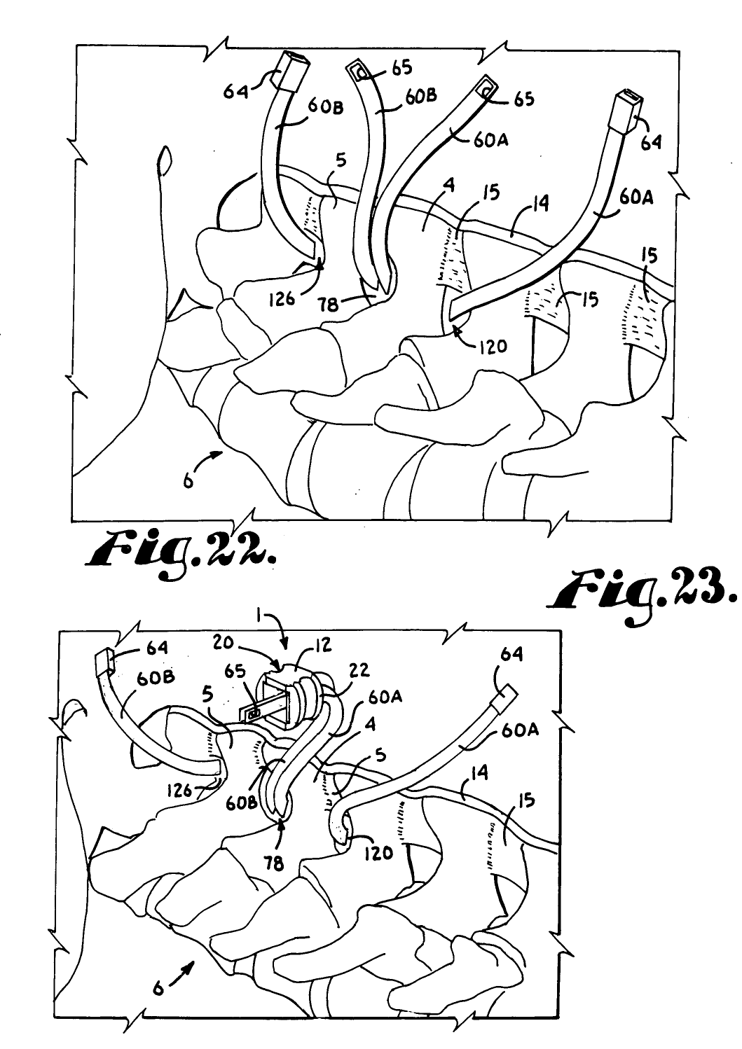

Specifically, a pair of identical bands or ribbons, 60A and 60B are illustrated in FIGS. 17-28. Each of the bands 60A and 60B is elongate and substantially flat, preferably having limited elasticity to allow for sufficient tightening about a spinous process and a portion of the device 1. The bands may be formed of a monofilament or woven, braided or otherwise formed, and prepared from a variety of materials including plastics, such as thermoplastic polymer resins, silastics including polyesters, for example, polyethylene terephthalate (PET). The bands 60A and 60B are sized and shaped to be received in the through channel 44 and fit snugly against both a spinous process and one of the interior curved walls 50 or 52 of the device 1. Each band 60A and 60B includes an attached self-locking, anti-slip fixing structure or buckle 64 on one end thereof and an aperture or eyelet 65 formed near an opposite end thereof. The locking structures 64 are known in the art, and for example, include a channel for extending the band 60 therethrough to form a loop about a portion of the device 1 and thereafter tighten the loop as will be described more fully below. Within the buckle channel, the locking structure or buckle 64 includes, for example, reverse angle teeth, hooks or pressure structure that prohibits the band 60 from feeding back through the buckle 64 once threaded into and through the buckle and tensioned, as illustrated in FIG. 26.

In use, an incision 70 is first made with a cutting tool 71 and held open with a tool 72 near the spinous processes 4 and 5. As illustrated in FIG. 6, according to methods of the invention, the incision 70 is advantageously located in the midline or somewhat laterally to the spine, on one side only; and it is not necessary to detach or section the supraspinous ligament 14 or the interspinous ligament 15 in advance of implantation of the device 1, thus providing a more minimally invasive procedure with muscle dissection and gentle soft tissue retraction limited to just one side.

With reference to FIG. 7, a cutting or shaving tool 74 having a rounded working surface 76 is then used to prepare an implantation cite by inserting the tool 74 into the incision and utilizing the working surface 76 to remove small portions of the spinous processes 4 and 5 and a lower portion 1SA of the interspinous ligament 15, if necessary, adjacent to an interspinous space, generally 78, into which the device 1 will eventually be implanted. The tool working surface 76 is rotated and/or otherwise maneuvered until a desired shape of interspinous space 78 results that substantially conforms to a desired form for engagement with the faces 20 and 22 of the device 1.

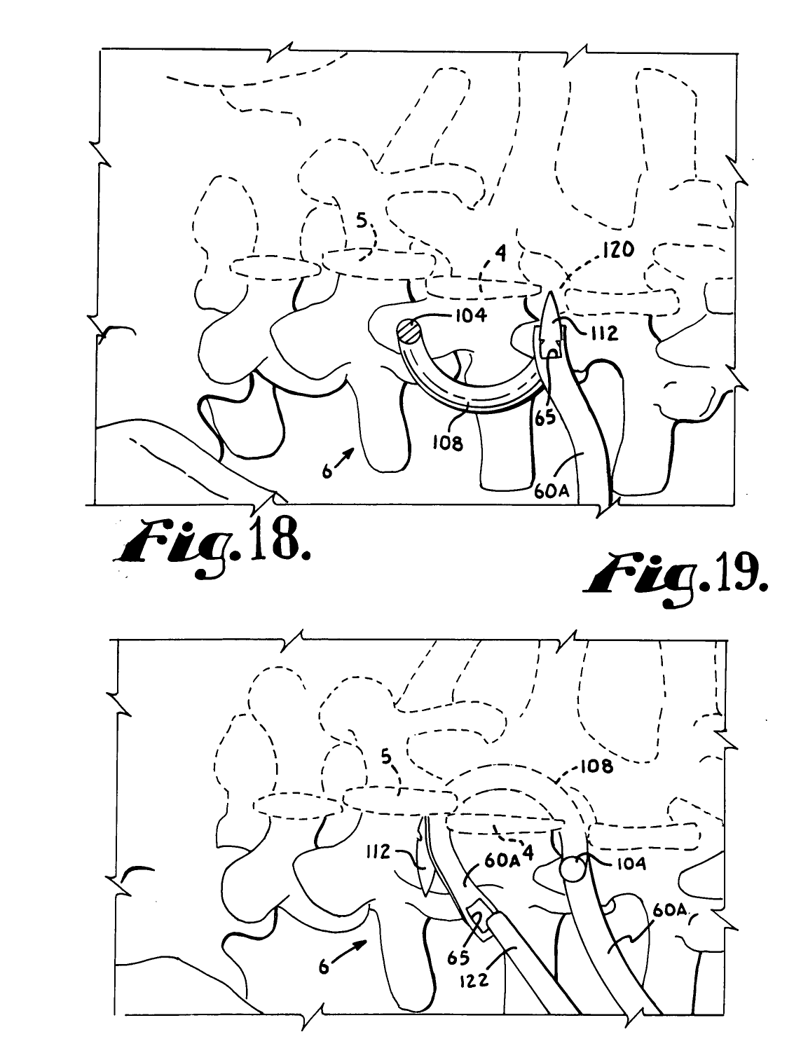

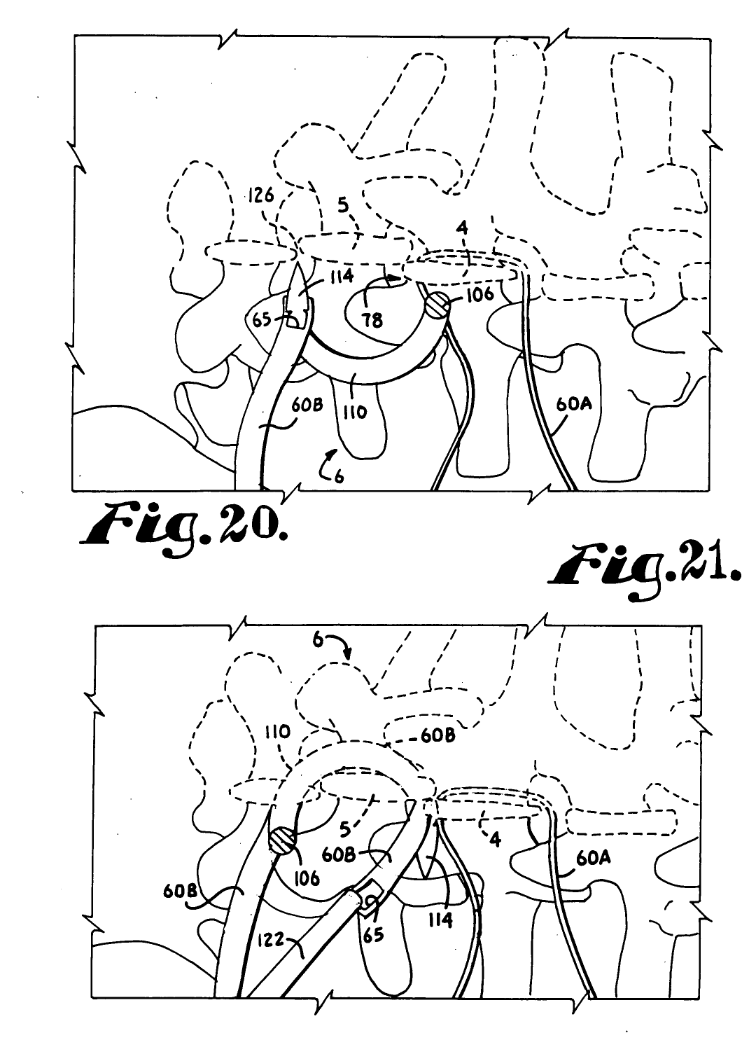

With particular reference to FIGS. 8-15, in order to determine an adequate size of device 1 to fit with a particular patient's interspinous process spacing and anatomy, one or more trial spacers 80 are tested. Each trial spacer 80 is substantially similar to a device 1 with the exception that in lieu of a curved through channel 44, the spacer 80 includes an aperture 82 formed in a lateral face 84. The surfaces forming the aperture 82 include a further recess 86 for receiving a knob 88 of an insertion tool 90. The tool 90 is elongate and includes a bend 92 to allow for lateral insertion of the trial spacer 80 into the interspinous space 78. A substantially block-shaped end portion 94 of the tool 90 is received in the aperture 82 with the knob 88 “snapping” or otherwise engaging into the recess 86 as illustrated in FIGS. 12 and 13. Then, as illustrated in FIGS. 14 and 15, the trial spacer 80 is carefully inserted into the interspinous space 78 and tested for correct fit. As illustrated in FIG. 8, each size of trial spacer may be identified with a numeral imprinted thereon to indicate size, and each available size of spacer 80 may be tested until a correct fit is achieved. A spacer device 1 of the invention is then chosen that is of the same size as the chosen trial spacer 80. Each trial spacer 80 has an identical aperture 82 such that the same insertion tool 90 may be used with each sized trial spacer 80.

With particular reference to FIGS. 16-22, after the trial spacer 80 is removed, the bands 60A and 60B are each implanted at the cite 78. With reference to FIG. 16, the bands are implanted utilizing the band insertion tools 96 and 98. The tools 96 and 98 include respective handles 100 and 102; respective elongate shafts 104 and 106; respective curved hook portions 108 and 110 disposed perpendicular to the respective shafts 104 and 106; and respective pointed tips 112 and 114 terminating the respective hook portions 108 and 110. The band insertion tools 96 and 98 are identical with the exception of a direction of curvature of the hook portions 108 and 110. The hook portion 108 curves in a counterclockwise direction from the shaft 104 substantially forming a half circle and the hook portion 110 curves in a clockwise direction from the shaft 106 substantially forming a half circle.

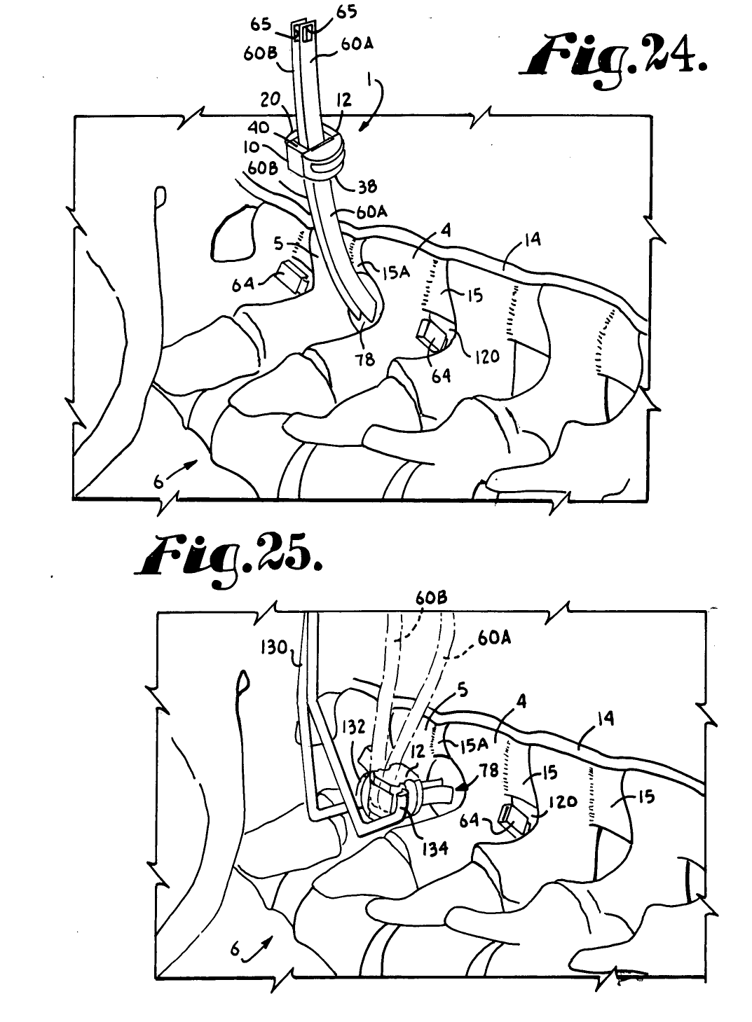

With reference to FIGS. 17-19, the tool 96 is utilized to thread the band 60A about the spinous process 4 and through the space or cite 78. The pointed tip 112 is inserted into the eyelet 65 of the band 60A and the tip 112 with the threaded band 60A are inserted into a space 120 located adjacent to the spinous process 4 on the other side of the prepared space 78. The handle 100 of the tool 96 is then rotated, rotating the shaft 104 and in particular the semi-circular hook portion 108 in a counterclockwise direction about the spinous process 4, threading the band 60A through the interspinous space 78. With reference to FIG. 19, a grasping tool, such as a forceps 122 is used to seize and hold the band 60A, pulling the band 60A and turning the band 60A such that a flat surface thereof is disposed about the spinous process 4 as illustrated in FIG. 20. The handle 100 of the tool 96 is then rotated in a clockwise manner to turn the hook portion 108 back out of the interspinous space 78 and then out of the space 120.

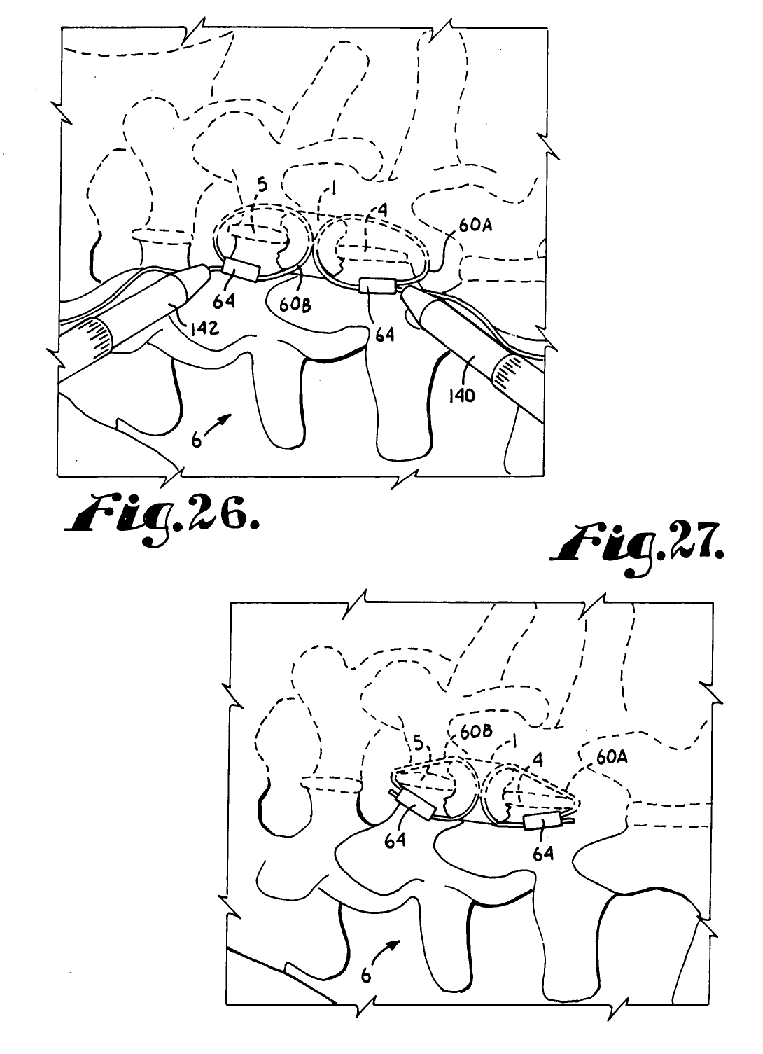

With reference to FIGS. 20-21, the band 60B is implanted in a method similar to that described above with respect to the band 60A. The pointed tip 114 is inserted into the eyelet 65 of the band 60B and the tip 114 with the threaded band 60B are inserted into a space 126 located adjacent to the spinous process 5 on the other side of the prepared space 78. The handle 102 of the tool 98 is then rotated, rotating the shaft 106 and in particular the semi-circular hook portion 110 in a clockwise direction about the spinous process 5, threading the band 60B through the interspinous space 78. With reference to FIG. 21, a grasping tool, such as the forceps 122 is used to seize and hold the band 60B, pulling the band 60B and turning the band 60B such that a flat surface thereof is disposed about the spinous process 5 as illustrated in FIG. 22. The handle 102 of the tool 98 is then rotated in a counterclockwise manner to turn the hook portion 110 out of the interspinous space 78 and then the space 126.

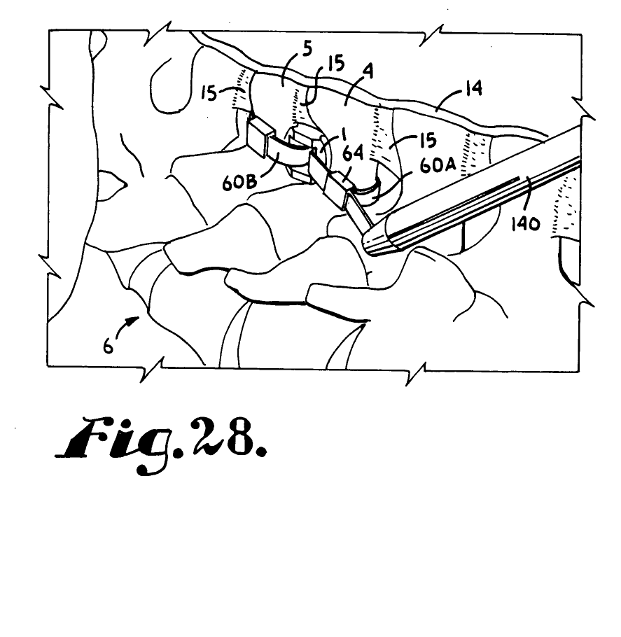

With further reference to FIG. 22 and with reference to FIG. 23, the bands 60A and 60B are pulled to a desired position with the eyelets 65 of the bands aligned after which both bands 60A and 60B are threaded into the through channel 44 of the device 1 at the lateral insertion face 38 and toward the trailing face 40, the flat surfaces of the bands being adjacent to the curved walls 50 and 52 defining the channel 44. With reference to FIG. 24, as the bands 60A and 60B are pulled through the channel 44, the fixing structures or buckles 64 are pulled into position adjacent the interspinous spaces 120 and 126. With reference to FIG. 25, a spacer holding tool 130 is used to hold and insert the spacer device 1 into the cite 78 utilizing prongs 132 and 134 that engage respective tool grooves 34 and 34A on the device 1.

With reference to FIGS. 26-28, once the device 1 is inserted into the cite 78 with the faces 20 and 22 engaging the spinous processes 5 and 4 respectively, a pair of band grasping tools 140 and 142 are utilized to thread the bands 60A and 60B through respective buckles 64 thereon and tighten the bands, the buckles 64 locking the bands snugly in place about the spinous processes 4 and 5 and surfaces of the spacer device 1. The bands 60A and 60B are then trimmed.

Removal of the device 1, if necessary, includes the following steps: cutting the bands 60A and 60B; removing the band portions from the interspinous spaces 120 and 126; and then removing the device 1 from the interspinous space 78.

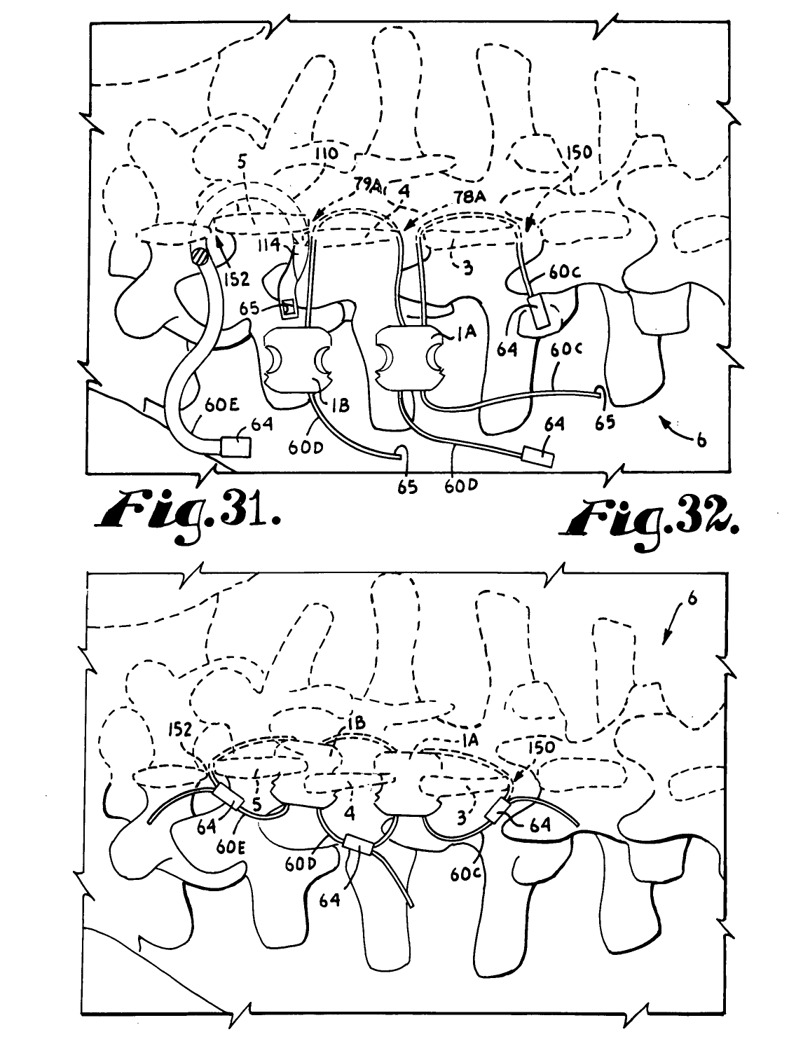

With reference to FIGS. 29-33, two or more devices 1 according to the invention may be implanted according to a method of the invention into adjacent interspinous spaces. The implantation procedure is similar to that described previously herein with respect to a single device 1. The example illustrated in FIGS. 29-33 and described herein includes two devices 1A and 1B that are identical to the device 1 previously described herein and also bands 60C, 60D and 60E identical to the bands 60, 60A and 60B previously described herein. For this example, the same human spine 6 is illustrated, along with spinous processes 3, 4, and 5. Although not shown in the drawings, similar to what was previously described herein, interspinous process spaces 78A and 79A are first prepared utilizing the tool 74 shown in FIG. 7. Trial spacers 80 are then inserted into the spaces to determine a correctly sized spacer 1A and a correctly sized spacer 1B as illustrated in FIGS. 14 and 15 and previously described herein.

With reference to FIG. 29, after removal of the trial spacer 80, the tool 96 is utilized to thread the band 60C about the spinous process 3 and through the space or cite 78A. The pointed tip 112 is inserted into the eyelet 65 of the band 60C and the tip 112 with the threaded band 60C are inserted into a space 150 located adjacent to the spinous process 3 on the other side of the prepared space 78A. The handle 100 of the tool 96 is then rotated, rotating the shaft 104 and in particular the semi-circular hook portion 108 in a counterclockwise direction about the spinous process 3, threading the band 60C through the interspinous space 78A. Similar to what is shown in FIG. 19, a grasping tool, such as a forceps 122 is used to seize and hold the band 60C, pulling the band 60C and turning the band 60C such that a flat surface thereof is disposed about the spinous process 3 as illustrated in FIG. 30. The handle 100 of the tool 96 is then rotated in a clockwise manner to turn the hook portion 108 back out of the interspinous space 78A and then out of the space 150. The band 60C is then threaded through the channel 44 of the device 1A by inserting the eyelet 65 end into the channel 44 at the leading face 38 and out of the trailing face 40 as illustrated in FIG. 30.

Also with reference to FIG. 30, the tool 96 is again utilized to thread the band 60D about the spinous process 4 and through the space or cite 79A. However, before insertion, the band 60D is threaded through the device 1A by inserting the end of the band 60D having the eyelet 65 into the device 1A at the trailing face 40, through the channel 44 and out of the leading face 38. The pointed tip 112 of the tool is then inserted into the eyelet 65 of the band 60D and the tip 112 with the threaded band 60D are inserted into the prepared space 78A. The handle 100 of the tool 96 is then rotated, rotating the shaft 104 and in particular the semi-circular hook portion 108 in a counterclockwise direction about the spinous process 4, threading the band 60D through the interspinous space 79A. Similar to what is shown in FIG. 19, a grasping tool, such as a forceps 122 is used to seize and hold the band 60D, pulling the band 60D and turning the band 60D such that a flat surface thereof is disposed about the spinous process 4 as illustrated in FIG. 31. The handle 100 of the tool 96 is then rotated in a clockwise manner to turn the hook portion 108 back out of the interspinous space 79A and then out of the space 78A. The band 60D is then threaded through the channel 44 of the device 1B by inserting the eyelet 65 end into the channel 44 at the leading face 38 and out of the trailing face 40 as illustrated in FIG. 31.

Also with reference to FIG. 31, the band 60E is implanted in a method similar to that described above with respect to the bands 60C and D, but with the tool 98. The pointed tip 114 is inserted into the eyelet 65 of the band 60E and the tip 114 with the threaded band 60E are inserted into a space 152 located adjacent to the spinous process 5 on the other side of the prepared space 79A. The handle 102 of the tool 98 is then rotated, rotating the shaft 106 and in particular the semi-circular hook portion 110 in a clockwise direction about the spinous process 5, threading the band 60E through the interspinous space 79A. A grasping tool, such as the forceps 122 is used to seize and hold the band 60E, pulling the band 60E and turning the band 60E such that a flat surface thereof is disposed about the spinous process 5. The handle 102 of the tool 98 is then rotated in a counterclockwise manner to turn the hook portion 110 out of the interspinous space 79A and then the space 152. The band 60E is then threaded through the channel 44 of the device 1B by inserting the eyelet 65 end into the channel 44 at the leading face 38 and out of the trailing face 40.

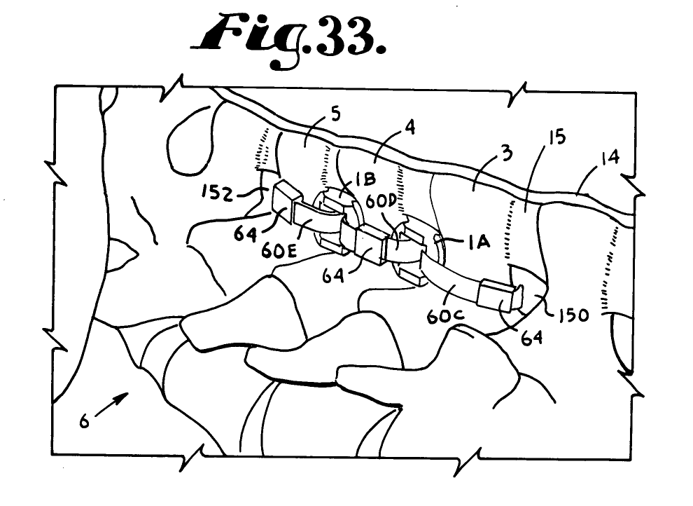

With reference to FIG. 32, the bands 60C, 60D and 60E are pulled through the channels 44 of the devices 1A and 1B, and the fixing structures or buckles 64 of each of the bands 60C and 60E are pulled into position adjacent the interspinous spaces 150 and 152. The spacer holding tool 130 is used to hold and insert the spacer device 1A into the cite 78A utilizing prongs 132 and 134 that engage respective tool grooves 34 and 34A on the device 1A. The spacer holding tool 130 is also used to hold and insert the spacer device 1B into the cite 79A utilizing prongs 132 and 134 that engage respective tool grooves 34 and 34A on the device 1B.

With reference to FIGS. 32-33, band grasping tools 140 and 142 are then utilized to thread the bands 60C, 60D and 60E through respective buckles 64 thereon and tighten the bands, the buckles 64 locking the bands snugly in place about the respective spinous processes 3, 4 and 5 and surfaces of the spacer devices 1A and 1B. The bands 60C, 60D and 60E are then trimmed.

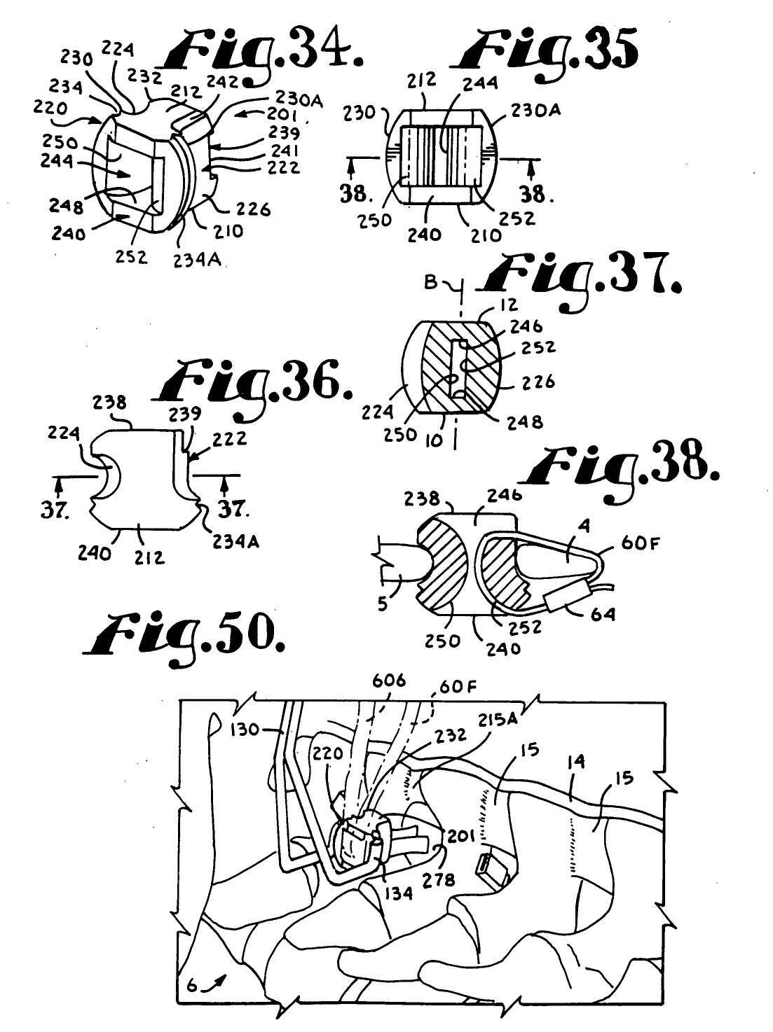

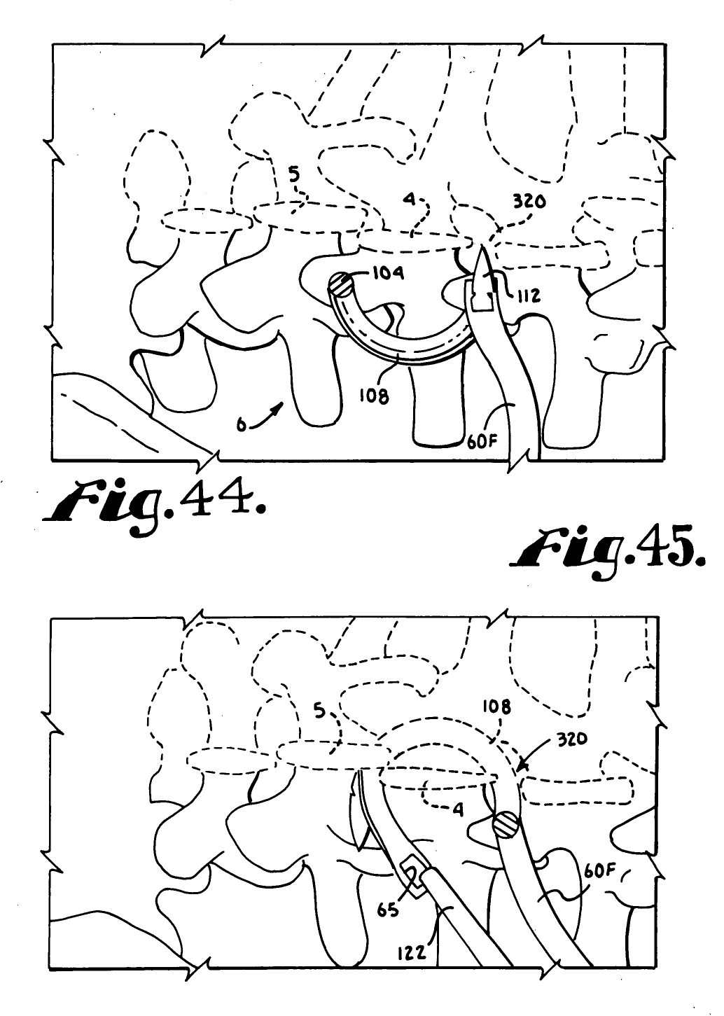

With reference to FIGS. 34-53, the reference numeral 201 generally designates an alternative embodiment of an interspinous process spacer device of the present invention. The device 201 is also used to maintain proper spacing between a pair of spinous processes 4 and 5 of a human spine, preferably a lower portion thereof, generally 6. The device 201 is preferably constructed from a single, unitary blank or molded, strong structure. The device 201 may be rigid or somewhat elastic and may be made from metals, metal alloys, plastics and/or composites. For example, the device 201 may be molded or machined from a plastic material such as polyetheretherketone (PEEK). Devices 201 according to the invention are made in incremental sizes so that a desired size of device 201 is implanted for cooperating with a particular patient's spine 6.

In particular, the illustrated device 201 has a substantially flat anterior surface 210 for facing toward vertebrae 4V and 5V of the lower spinal portion 6 and a substantially flat posterior surface 212 opposite the surface 210 for facing away from the vertebrae and toward the supraspinous ligament 14 and a portion of interspinous ligament 15 that is disposed between the pair of spinous processes 4 and 5, the surface 212 being identical or substantially similar to the surface 210. The surfaces 210 and 212 are disposed in substantially parallel planes.

The device 201 further includes an inferior or lower saddle-like abutment face or portion, generally 220, and an opposite superior or upper abutment face or portion, generally 222 that is not saddle-like in nature, thus providing an asymmetric device having lateral implantation advantages to be discussed in greater detail below. The portion or face 220 is substantially arcuate and includes an arcuate U-shaped furrow or channel 224 defined in part by arcuate rims 230 and 232. The portion or face 222 includes a slightly arcuate face 226 and an arcuate rim 230A that is similar to the rim 230. Unlike the rim 232, the portion or face 222 does not include a second or leading rim. The absence of such a rim aids in implanting the device 201, wherein the device 201 may be rotated or turned into place between the spinous processes 4 and 5, requiring less preparation and removal of bone, ligament and other body tissue around an interspinous space 278 that is the implantation cite for the device 201. In order to provide stability and structure to keep a band 60F and attached device 201 in place with respect to the spinous process 4, a portion of the face 222 and a leading face 238 includes a depression or squared-off notch 239 sized and shaped such that a width of the band 60F fits within the notch 239, the notch further defined by a flat side 241 so that a flat surface of the band 60F fits snugly against the device 201 at the notch 239.

Each of the arcuate portions 224, 226, 230, 232 and 230A have a substantially convex outer profile, curving substantially uniformly outwardly from the opposed surfaces 210 and 212. The U-shaped channel 224 formed in the face 220 is substantially uniform and centrally located and otherwise sized and shaped for engagement with the spinous processes 5. The channel 224 is non-linear, having a substantially convex outer profile running from the surface 210 to the surface 212. Formed in the rims 230 and 230A are tool receiving grooves 234 and 234A, respectively. Although grooves 234 and 234A are shown that run from the surface 210 to the surface 212, it is foreseen that other types of tool receiving formations may be used including but not limited to notches, apertures and the like.

The rim 232 and the notch 239 are each adjacent to the lateral insertion or leading face or portion, generally 238 that is disposed opposite a lateral trailing face or portion, generally 240. The grooved rims 230 and 230A are disposed near the face 240. Both faces 238 and 240 are preferably beveled or rounded to aid in inserting the device 201 between the spinous processes 4 and 5. The surface 212 also includes a bevel 242. A band receiving channel 244 extends through the device 201 between the lateral faces 238 and 240. The channel is further defined by a pair of opposed substantially flat walls 246 and 248 and a pair of arcuate walls 250 and 252. The walls 246 and 248 are substantially parallel to one another and to the anterior and posterior surfaces 210 and 212. The walls 250 and 252 curve towards one another, but are not mirror images as shown in FIG. 38 due to the presence of the notch 239 that guides and controls the location of the band 60F as described above. As illustrated in FIG. 37, at a smallest opening in an interior of the device 201, the walls defining the channel 244 form an opening having a substantially rectangular cross-section, a length of the rectangular cross-section extending along a substantially central axis B. The channel 244 then widens in a direction toward the face 238 and also in an opposite direction toward the face 240 as best illustrated in FIG. 38.

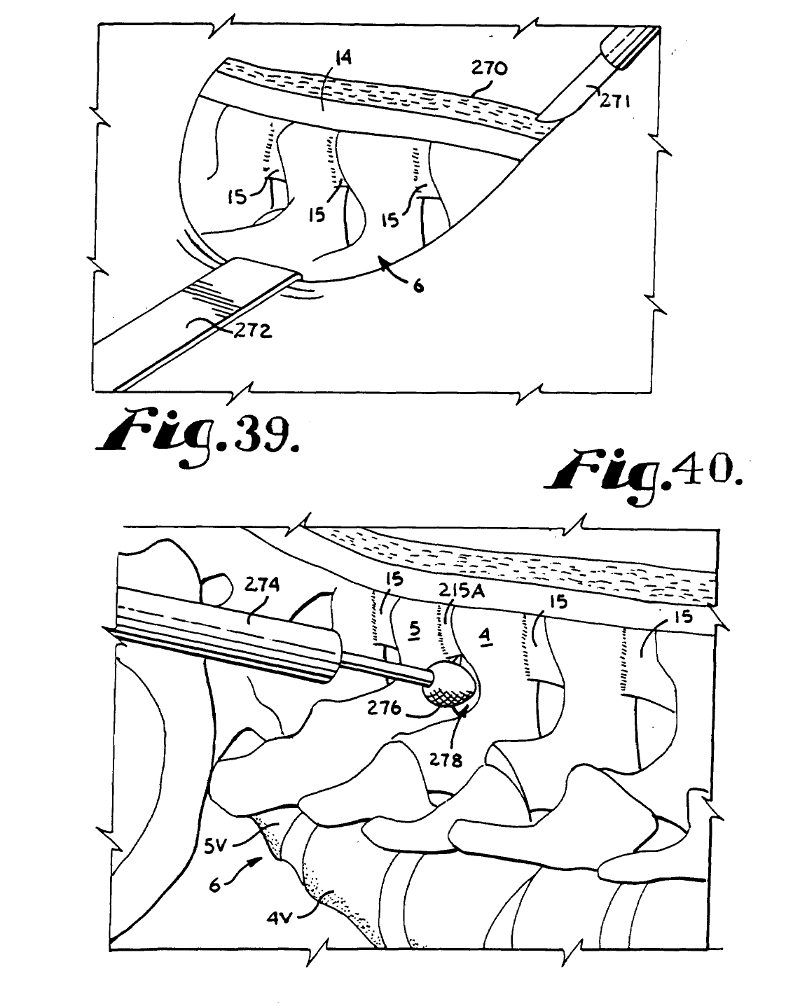

One or more devices 201 according to the invention are implantable between pairs of spinous processes attaching to adjacent spinous processes utilizing bands, ties or tapes, generally 60 as described previous herein and as shown as ties 60F and 60G in the drawing figures. With reference to FIG. 39, in use, an incision 270 is first made with a cutting tool 271 and held open with a tool 272 near the spinous processes 4 and 5. The incision 270 is advantageously located laterally to the spine, on one side only; and it is not necessary to detach or section the supraspinous ligament 14 or the interspinous ligament 15 in advance of implantation of the device 201. Thus providing a minimally invasive procedure.

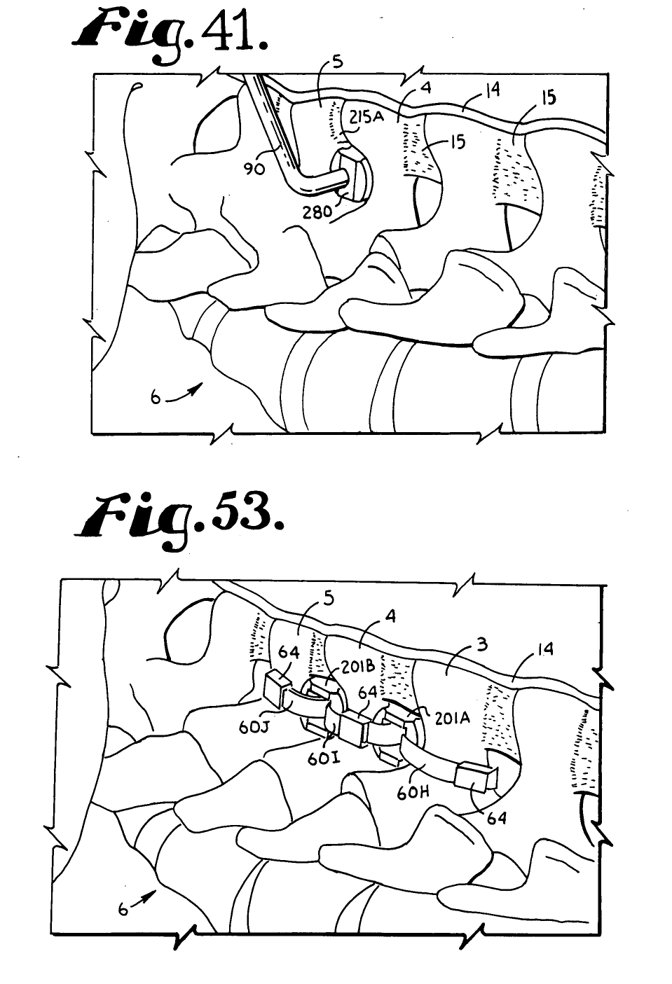

With reference to FIG. 40, a cutting or shaving tool 274 having a rounded working surface 276 is then used to prepare an implantation cite by inserting the tool 274 into the incision and utilizing the working surface 276 to remove small portions of the spinous processes 4 and 5 and a portion 215A of the interspinous ligament 15, if necessary, adjacent to an interspinous space, generally 278, into which the device 201 will eventually be implanted. The tool working surface 276 is rotated and/or otherwise maneuvered until a desired shape of interspinous space 278 results that substantially conforms to a desired form for engagement with the faces 220 and 222 of the device 201. As indicated above, as compared to the space 78 required for the device 1, the space 278 may be made slightly smaller, advantageously allowing for the removal of less bone and ligament in view of the asymmetrical geometry of the device 201 wherein a rim 32A is replaced by a notch 239.

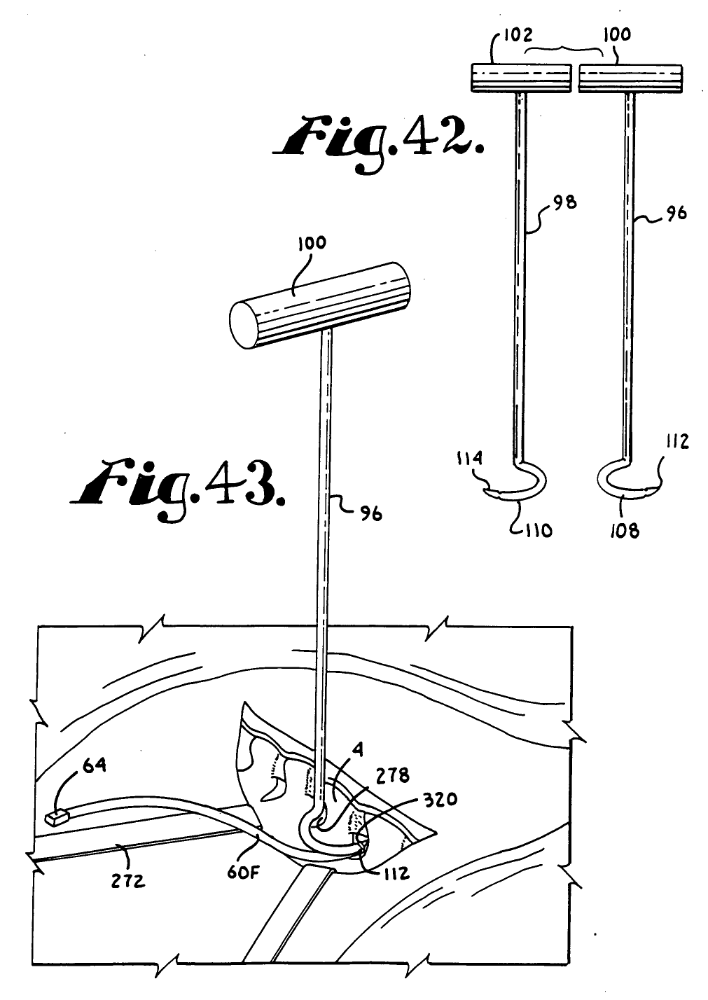

With reference to FIG. 41, in order to determine an adequate size of device 201 to fit with a particular patient's interspinous process, one or more trial spacers 280 are tested. Each trial spacer 280 is substantially similar to a device 201 with the exception that in lieu of a curved through channel 244, the spacer 280 includes an aperture similar or identical to the aperture 82 previously described herein with respect to the trial spacer 80 and the device 1. Thus the tool 90 cooperates with the spacer 280 as previously described herein to allow for measurement of the space 278 to determine the appropriately sized device 201.

With particular reference to FIGS. 42-47, after the trial spacer 280 is removed, the bands 60F and 60G are each implanted at the cite 278. With reference to FIGS. 16 and 42, the bands are implanted utilizing the band insertion tools 96 and 98 previously described herein.

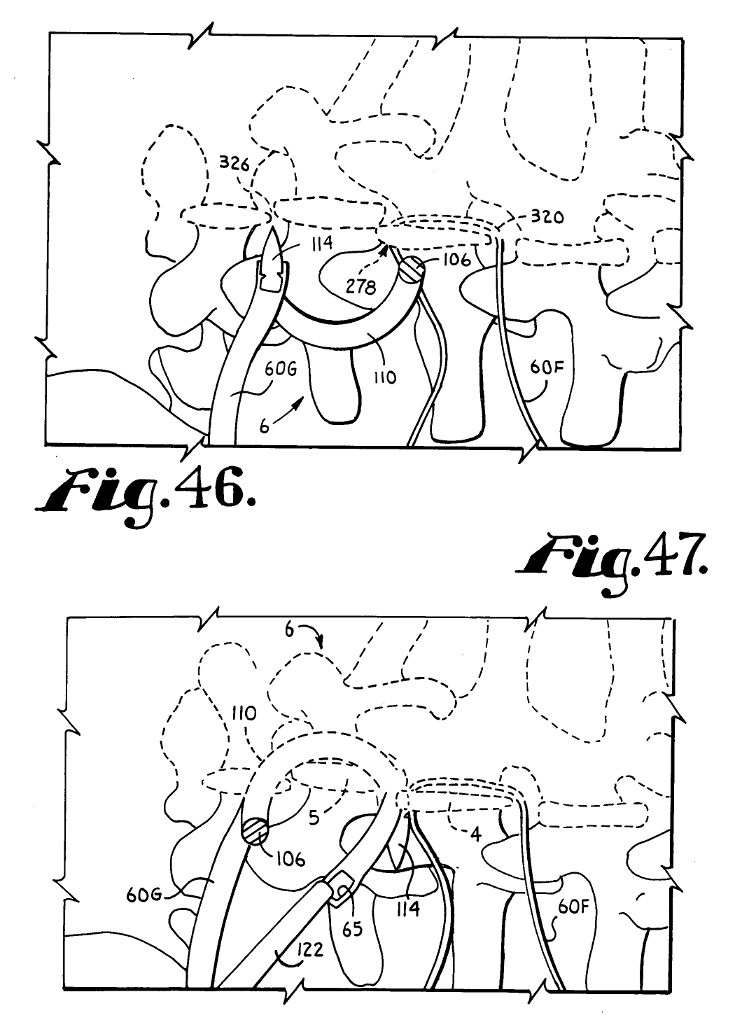

With reference to FIGS. 43-45, the tool 96 is utilized to thread the band 6OF about the spinous process 4 and through the space or cite 278. The pointed tip 112 is inserted into the eyelet 65 of the band 60F and the tip 112 with the threaded band 60F are inserted into a space 320 located adjacent to the spinous process 4 on the other side of the prepared space 278. The handle 100 of the tool 96 is then rotated, rotating the shaft 104 and in particular the semi-circular hook portion 108 in a counterclockwise direction about the spinous process 4, threading the band 60F through the interspinous space 278. With reference to FIG. 45, a grasping tool, such as the forceps 122 is used to seize and hold the band 60F, pulling the band 60F and turning the band 60F such that a flat surface thereof is disposed about the spinous process 4 as illustrated in FIG. 46. The handle 100 of the tool 96 is then rotated in a clockwise manner to turn the hook portion 108 back out of the interspinous space 278 and then out of the space 320.

With reference to FIGS. 46-47, the band 60G is implanted in a method similar to that described above with respect to the band 60G. The pointed tip 114 is inserted into the eyelet 65 of the band 60G and the tip 114 with the threaded band 66GB are inserted into a space 326 located adjacent to the spinous process 5 on the other side of the prepared space 278. The handle 102 of the tool 98 is then rotated, rotating the shaft 106 and in particular the semi-circular hook portion 110 in a clockwise direction about the spinous process 5, threading the band 60G through the interspinous space 278. With reference to FIG. 47, a grasping tool, such as the forceps 122 is used to seize and hold the band 60G, pulling the band 60G and turning the band 60G such that a flat surface thereof is disposed about the spinous process 5 as illustrated in FIGS. 47 and 48. The handle 102 of the tool 98 is then rotated in a counterclockwise manner to turn the hook portion 110 out of the interspinous space 278 and then the space 326.

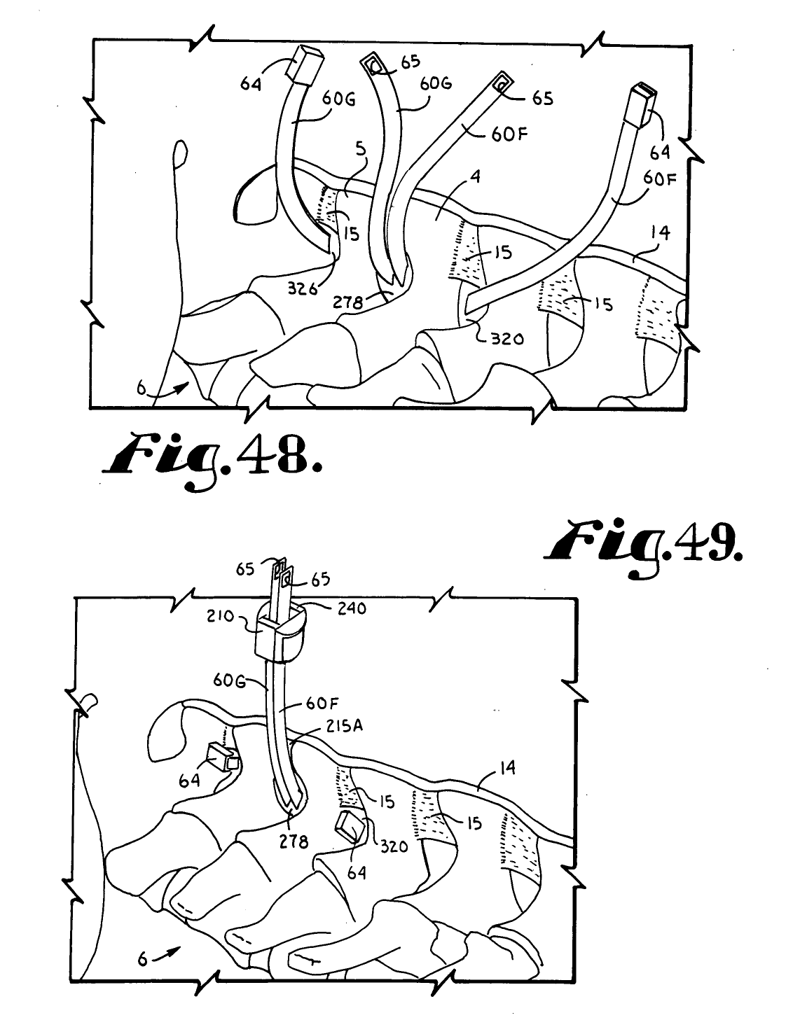

With further reference to FIG. 48 and with reference to FIG. 49, the bands 60F and 60G are pulled to a desired position with the eyelets 65 of the bands aligned after which both bands 60F and 60G are threaded into the through channel 244 of the device 201 at the lateral insertion face 238 and toward the trailing face 240, the flat surfaces of the bands being adjacent to the curved walls 250 and 252 defining the channel 244. With reference to FIG. 49, as the bands 60F and 60G are pulled through the channel 244, the fixing structures or buckles 64 are pulled into position adjacent the interspinous spaces 320 and 326. With reference to FIG. 50, the spacer holding tool 130 previously described herein is used to hold and insert the spacer device 201 into the cite 278 utilizing prongs 232 and 234 that engage respective tool grooves 234 and 234A on the device 201. As discussed above, the device 201 is inserted into the space 278 with the face 220 in the lead, being directed toward the spinous process 5, rather than moving the face 238 directly and evenly between the processes 4 and 5. Once the rim 232 is located in the space 278 near the spinous process 5, the device 201 is rotated into place, with the rim 230A being moved into place next to the spinous process 4. The device 201 is then in place as shown in FIG. 38.

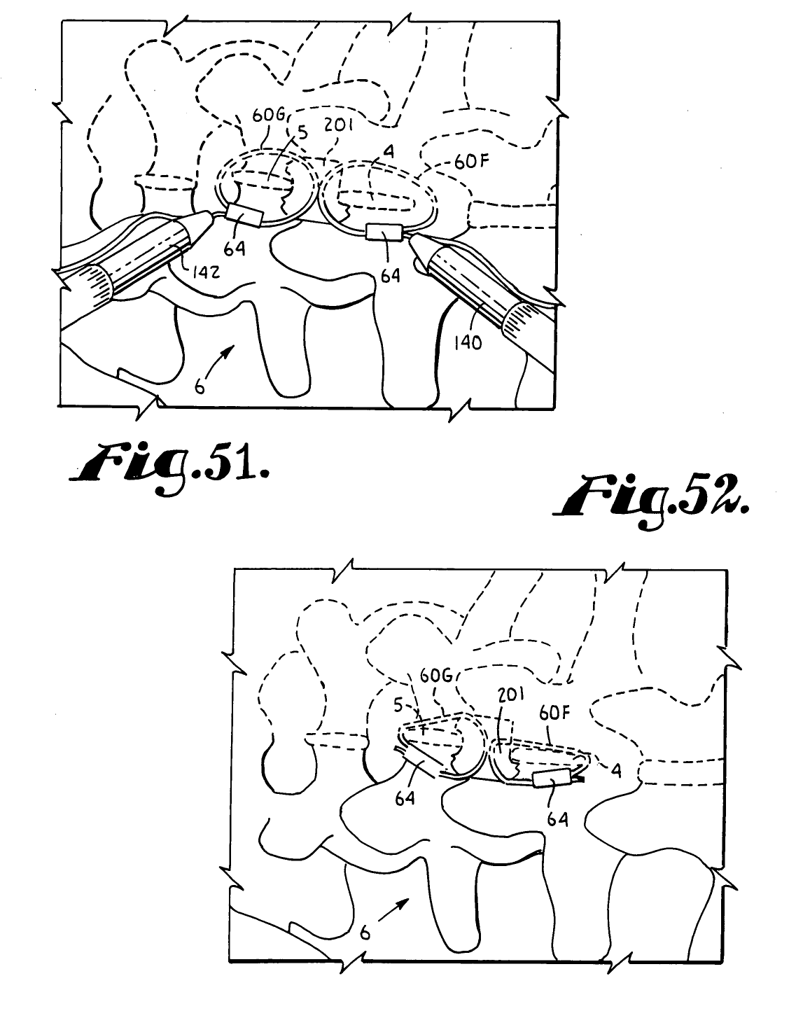

With reference to FIGS. 51-52, once the device 201 is inserted into the cite 278 with the faces 220 and 222 engaging the spinous processes 5 and 4 respectively, a pair of band grasping tools 140 and 142 are utilized to thread the bands 60F and 60G through respective buckles 64 thereon and tighten the bands, the buckles 64 locking the bands snugly in place about the spinous processes 4 and 5 and surfaces of the spacer device 201. The bands 60F and 60G are then trimmed.

Removal of the device 201, if necessary, includes the following steps: cutting the bands 60F and 60G; removing the band portions from the interspinous spaces 320 and 326; and then removing the device 201 in a rotating manner from the interspinous space 278, first removing the device at the rim 230A and rotating generally towards the spinous process 5.

With reference to FIG. 53 a pair of devices 201A and 201B are shown implanted between adjacent spinal processes 3, 4, and 5. The devices 201A and 201B are the same or substantially similar to the device 201 previously described herein. The devices 201A and 201B are implanted according to a method previously described herein with respect to the devices 1A and 1B and illustrated in FIGS. 29-33.

It is to be understood that while certain forms of the present invention have been illustrated and described herein, it is not to be limited to the specific forms or arrangement of parts described and shown.

What is claimed and desired to be secured by Letters Patent is as follows:

1. A device for placement between a pair of spinous processes, the device comprising:
(a) opposed abutment surfaces sized and shaped for engaging adjacent spinal processes;
(b) at least one side-to-side through channel disposed between the opposed abutment surfaces; and
(c) at least one band receivable in the through channel.

2. The device of claim 1 wherein at least one of the opposed abutment surfaces includes an arcuate furrow.

3. The device of claim 1 wherein the device is asymmetrical.

4. The device of claim 1 wherein one of the abutment surfaces has a centrally located arcuate furrow running substantially perpendicular to the channel.

5. A device for placement between a pair of spinous processes, the device comprising:
(a) arcuate opposed abutment surfaces sized and shaped for engaging adjacent spinal processes;
(b) a curved through channel disposed between the opposed abutment surfaces; and
(c) a pair of bands receivable in the curved through channel.

6. The device of claim 5 wherein at least one of the opposed abutment surfaces includes an arcuate furrow.

7. The device of claim 5 wherein the device is asymmetrical.

8. The device of claim 5 wherein one of the abutment surfaces has a centrally located arcuate furrow running substantially perpendicular to the channel.

9. In a device for placement between adjacent spinous processes having opposed abutment surfaces for engaging adjacent spinal processes and at least two bands for attaching the interbody device to the spinous processes, the improvement wherein:
the device defines a through channel located between the abutment surfaces and running substantially transverse to the abutment surfaces, the through channel receiving the at least two bands therethrough.

10. The improvement of claim 9 wherein at least one of the abutment surfaces includes an arcuate furrow.

11. The improvement of claim 9 wherein the device is asymmetrical.

12. The improvement of claim 9 wherein one of the abutment surfaces has a centrally located arcuate furrow running substantially perpendicular to the channel.

13. In a device for placement between adjacent spinous processes having opposed abutment surfaces for engaging adjacent spinal processes and at least two bands for attaching the interbody device to the spinous processes, the improvement wherein:
the device is asymmetrical, with one of the abutment surfaces defining a curvate furrow and the opposed abutment surfaces being curvate and smooth.

14. The improvement of claim 13 wherein the device defines a through channel located between the abutment surfaces and running substantially transverse thereto.

15. In a method of implanting an interspinous spacer, the improvement including the steps of:
a) making an incision;
b) unilaterally inserting the spacer between a pair of interspinous processes without detachment of the supraspinous ligament; and
c) securing the spacer to the pair of interspinous processes with a pair of bands.

16. In a method of implanting an interspinous spacer, the improvement including the steps of:
a) making an incision in skin located over the spine;
b) inserting the spacer between a pair of interspinous processes from one side of the spine without detachment of the supraspinous ligament; and
c) securing the spacer to the pair of interspinous processes with a pair of bands.
